(19) United States
(12) Patent Application Publication
(10) Pub. No.: US 2005/0123561 A1
(43) Pub. Date: Jun. 9, 2005

(54) Radio lan access authentication system

(76) Inventors: Mitsuo Honda, Tokyo (JP); Kazuhiro Matsuo, Kanagawa (JP); Yasuyuki Izumi, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 10/515,253

(22) PCT Filed: Nov. 20, 2002
(86) PCT No.: PCT/JP02/12125

(30) Foreign Application Priority Data
May 20, 2002 (JP) 2002-145132

(51) Int. Cl. A61K 39/12

(52) U.S. Cl. 424/199.1

(57) ABSTRACT

This invention provides a recombinant BCG vaccine transformed by an expression vector having polynucleotide coding for exogenous antigenic protein, characterized in that, the BCG vaccine is used for initial antigen stimulus in immune induction by plural antigen stimulations and also provides a method for the immune induction where the initial antigen stimulus is carried out by the said BCG vaccine and one or more additional antigenic stimulation(s) is/are carried out by non-BCG vaccine expressing the same antigenic protein.

### TECHNICAL FIELD

The invention of this application relates to BCG vaccine and utilization thereof. More particularly, the invention of this application relates to a recombinant BCG vaccine used for initial antigen stimulation in immune induction for prevention and treatment of various infectious diseases, cancer, etc. and to a method for induction of immunity in human beings or animals using the BCG vaccine.

### BACKGROUND ART

Attenuated BCG strain of Mycobacterium bovis (hereinafter, referred to as “BCG”) is well known and is the most commonly used live bacterial vaccine due to its safety.

On the other hand, as a result of development and improvement in genetic recombination technique in the past two decades, there have been vigorous studies where microbes such as virus and bacteria are modified so that they express exogenous antigenic protein and are applied as vaccine vector for prevention and therapy of various infectious diseases and cancer. With regard to BCG, there have been reported recombinant BCG vaccines where human immunodeficiency virus (HIV) or simian immunodeficiency virus (SIV) is the target (J. Immunol. 164:4968-4978, 2000; J. Virol. 71:2303-2309, 1997; Infect. Immun. 57:283-288, 1989). Vaccine where HIV gene is expressed in BCG is able to induce immunity for a long period (at least for two years) and this immunity-inducing ability for such a long period is an excellent characteristic which is not noted in other DNA vaccines.

However, in the case of the conventional recombinant BCG vaccines, they are not always sufficient ability to induce immunity to the infectious disease, cancer, et al which is the target. For example, when immune induction is carried out to guinea pigs using a recombinant BCG vaccine using HIV-1 as a target, it is necessary to administer the dose which is from 50 to 100-fold of the usual dose (0.05-0.1 mg) of conventional BCG vaccine to human beings (Proc. Natl. Acad. Sci. USA 92:10698-10697, 1995). Further, in a test using macaque models, administration of only recombinant BCG for prevention from infection of pathogenic virus did not give favorable results.

Recombinant BCG vaccine is an excellent candidate for vaccine in view of its duration of effective immunity, safety and ease of production, and its effective utilization is urgently needed in the medical field.

The invention of this application has been achieved in view of the above-mentioned circumstances and has an object of providing a novel means for an effective utilization of recombinant BCG vaccine.

### DISCLOSURE OF THE INVENTION

As the first invention for solving the above-mentioned problems, this application provides a recombinant BCG vaccine transformed with an expression vector having a polynucleotide encoding an exogenous antigenic protein, which the BCG vaccine is used for initial antigen stimulation in immune induction by plural antigen stimulations.

As the second invention, this application provides a method for the immune induction by plural stimulations of exogenous antigenic protein, which comprises carrying out the initial antigen stimulation by the BCG vaccine of claim 1, and carrying out one or more additional antigenic stimulations by non-BCG vaccine expressing the same antigenic protein.

In the method of the second invention, it is a preferred embodiment that the vaccine for additional antigenic stimulation is a recombinant vaccinia virus vaccine such as recombinant DIs vaccine.

In addition, in the first and second inventions, it is a preferred embodiment that the antigenic protein is derived from immunodeficient virus or, to be more specific, the antigenic protein of immunodeficient virus is an HIV gene product such as Gag.

Thus, in accordance with the method of this invention, the virus acquired by a single administration of, for example, recombinant vaccinia virus (such as recombinant DIs-gag vaccine) can be prevented almost completely from flowing out into the blood and a decrease in CD4 cells can be suppressed as well. As a result, it is now possible to suppress the spread of at least pathogenic virus in the body and to prevent the progress of infectious diseases.

### BRIEF DESCRIPTION OF THE DRAWINGS

FIG. 1 is a schematic chart exemplifying the constitution of expression vector pSO-SIVgag used for the preparation of the recombinant BCG strain (rBCG-SIVgag) in Example 1.

FIG. 2 is the result of a Western blot analysis where amount of Gag protein produced from rBCG-SIVgag was measured.

FIG. 3 shows the change with the lapse of time of the number of copies of viral RNA (left drawing) and the change of CD4 cell counts with the lapse of time (right drawing) in blood of a control macaque when infected with pathogenic virus.

FIG. 4 shows the change with the lapse of time of the number of copies of viral RNA (left drawing) and the change with the lapse of time of CD4 cell counts (right drawing) in blood after infection with pathogenic virus in a macaque where rBCG-SIVgag only was vaccine-inoculated one time.

FIG. 5 shows the change with the lapse of time of the number of copies of viral RNA (left drawing) and the change with the lapse of time of CD4 cell counts (right drawing) in blood after infection with pathogenic virus in a macaque where rDIs-SIVgag only was vaccine-inoculated one time.

FIG. 6 shows the change with the lapse of time of the number of copies of viral RNA (left drawing) and the change with the lapse of time of CD4 cell counts (right drawing) in blood after infection with pathogenic virus in a macaque where rDIs-SIVgag+rBCG-SIVgag was vaccine-inoculated.

FIG. 7 shows the change with the lapse of time of the number of copies of viral RNA in blood after infection with pathogenic virus in a macaque where rBCG-SIVgag+rDIs-SIVgag was vaccine-inoculated. FIG. 7/1 shows the change with the lapse of time of CD4 cell count in blood after infection with pathogenic virus in a macaque where rBCG-SIVgag+rDIs-SIVgag was vaccine-inoculated.

FIG. 8 shows the change with the lapse of time of the number of copies of viral RNA in blood after inoculation with control vaccine (vector) to a macaque having a BCG anamnestic reaction. FIG. 8/1 shows the change with the lapse of time of CD4 cell count after inoculation with control vaccine (vector) to a macaque having a BCG anamnestic reaction.

FIG. 9 shows the change with the lapse of time of the number of copies of viral RNA in blood after vaccine-inoculation of rBCG-SIVgag (oral)+rDIs-SIVgag (intravenous) to a macaque having a BCG anamnestic reaction. FIG. 9/1 shows the change with the lapse of time of CD4 cell count after vaccine-inoculation of rBCG-SIVgag (oral)+rDIs-SIVgag (intravenous) to a macaque having a BCG anamnestic reaction.

FIG. 10 shows the change with the lapse of time the number of copies of viral RNA in blood after vaccine-inoculation with rBCG-SIVgag (intravenous)+rDIs-SIVgag (intravenous) to a macaque having a BCG anamnestic reaction. FIG. 10/1 shows the change with the lapse of time of CD4 cell count in blood after vaccine-inoculation with rBCG-SIVgag (intravenous)+rDIs-SIVgag (intravenous) to a macaque having a BCG anamnestic reaction.

### BEST MODE FOR CARRYING OUT THE INVENTION

The first invention is a recombinant BCG vaccine which is transformed by an expression vector having polynucleotide encoding exogenous antigenic protein. In immune induction by antigenic stimulation plural times, this recombinant BCG vaccine is characterized in that its use is for the initial antigen stimulation. Thus, the inventors of this application have found that, even in the case of recombinant BCG vaccine having insufficient immunity-inducing ability when only it is used, it enhances a specific immunity when it is used as a initial antigen stimulus (priming) followed by additional antigen stimuli (boosting), whereupon the present invention has been achieved.

With regard to the BCG strain, it is possible to use widely known ones which have been used for vaccination of tuberculosis, etc. With regard to the expression vector, it is possible to use a vector for BCG (such as plasmid pSO246) which has been used for the preparation of conventional recombinant BCG vaccine. When polynucleotide coding for the desired antigenic protein which is exogenous (in other words, not one of BCG) is inserted into a cloning site of this vector, it is possible to construct the expression vector. Incidentally, in the following description, exogenous antigenic protein may be referred to as “exogenous polypeptide” while polynucleotide coding for it may be referred to as “exogenous polynucleotide”. In addition, any promoter and terminator sequences derived from BCG strain (such as promoter and terminator sequences of heat shock protein (HSP) derived from BCG) are ligated to the polynucleotide, whereupon the exogenous polypeptide is well expressed.

An exogenous polynucleotide is a polynucleotide (such as cDNA fragment) which codes for antigenic protein other than one of a BCG strain. Anything may be used as the exogenous polypeptide as long as it brings about an antigen-antibody reaction in vivo. To be more specific, gag precursor p55 or p24 protein, env protein gp120 or gp160, pol precursor protein, nef protein, tat protein, etc. which are proteins of human immunodeficiency virus (HIV) which is a virus causing human acquired immune deficiency syndrome (AIDS) may be used as objects. It is also possible to use a similar antigenic polypeptide derived from simian immunodeficiency virus (SIV). It is further possible to use a polynucleotide coding for antigenic protein of cancer cells or other pathogens (other pathogenic viruses and bacteria).

With regard to a method for obtaining the exogenous polynucleotide, the significant sequence of the polynucleotide is cut out by an appropriate restriction enzyme from genome gene coding for exogenous polypeptide or cloned plasmid cDNA, or it is amplified by a polymerase chain reaction (PCR) using primer of an appropriate sequence. When it is not cloned, it is possible to obtain the above by amplification of DNA fragment by means of the above PCR using genomic DNA of animals or cells having that gene or, in the case of virus, using DNA or RNA derived from animal cells infected with virus as a template.

The expression vector constructed as such is introduced into BCG strain by known methods such as a calcium chloride method or an electroporation method and expression of the exogenous polypeptide of a transgenic microorganism is confirmed by a western blotting or by known immunological measuring method (such as ELISA) whereby the recombinant BCG of this invention can be prepared.

When the recombinant BCG thus prepared is suspended in a liquid carrier which is similar to that in the case of usual BCG vaccine, a recombinant BCG vaccine can be prepared and the resulting vaccine is able to be actually used for an immune induction method of the second invention.

A method of the second invention is characterized in that the initial antigen stimulus is carried out by the BCG vaccine of the above-mentioned first invention and one or more additional antigenic stimulation(s) is/are carried out by non-BCG vaccine expressing the same antigenic protein.

Vaccine for the additional antigenic stimulation (booster vaccine) can be prepared by transformation of known viruses or bacteria used for recombinant vaccine (such as poliovirus, influenza virus, rhinovirus, varicella virus, vaccinia virus, Salmonella bacteria and Listeria bacteria) with the same exogenous polynucleotide as the recombinant BCG vaccine (prime vaccine) of the first invention. In a method of the invention, a recombinant vaccinia virus DIs vaccine which was previously developed (Japanese Patent Laid-Open No. 20002/017370) by the inventors of this application is a preferred booster vaccine.

Administration of prime vaccine and booster vaccine can be carried out by known methods such as injection or oral administration. Although the dose and the schedule may be different depending upon type (human being or animal), body weight, type of the immunity to be induced, etc. of the individual to be inspected, prime vaccine may be 0.01 to 10 mg and booster vaccine may be 10⁵ to 10¹⁰ PFU for example. The time interval between inoculations of vaccine may be 3 to 12 months.

The invention of this application will now be illustrated as hereunder in more detail and specifically by way of the following Examples although the invention of this application is not limited by the following examples.

### EXAMPLES

### Example 1

### Preparation of Recombinant BCG

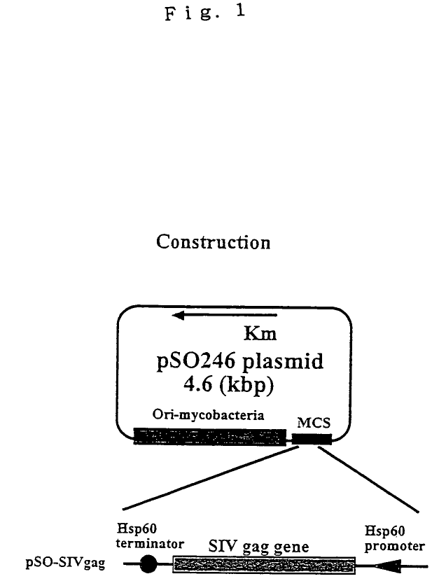

SIV gag gene was isolated from a plasmid pNL432 (J. Virol. 59:284-291, 1986), hsp60 promoter derived from BCG strain and terminator were ligated to front and rear of the said gene DNA, respectively and that is inserted into a multicloning site of a shuttle vector pSO246 of Escherichia coli-BCG strain (FEMS Microbiol. Lett. 135:237-243, 1996) whereupon an expression vector pSO-SIVgag was constructed (FIG. 1).

The expression vector was introduced into BCG Tokyo strain using Gene-pulser (Bio-Rad) according to a published method (Proc. Natl. Acad. Sci. USA 85:6987-6991, 1988) and the transformant was selected on a Middlebrook 7H10 agar medium (Difco) containing 20 μg/ml of kanamycin to prepare a recombinant BCG strain (rBCG-SIVgag) having pSO-SIVgag.

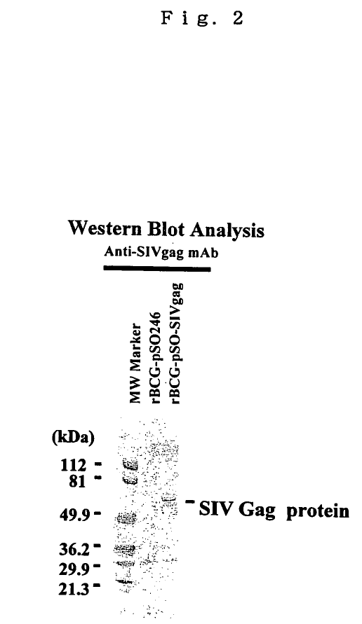

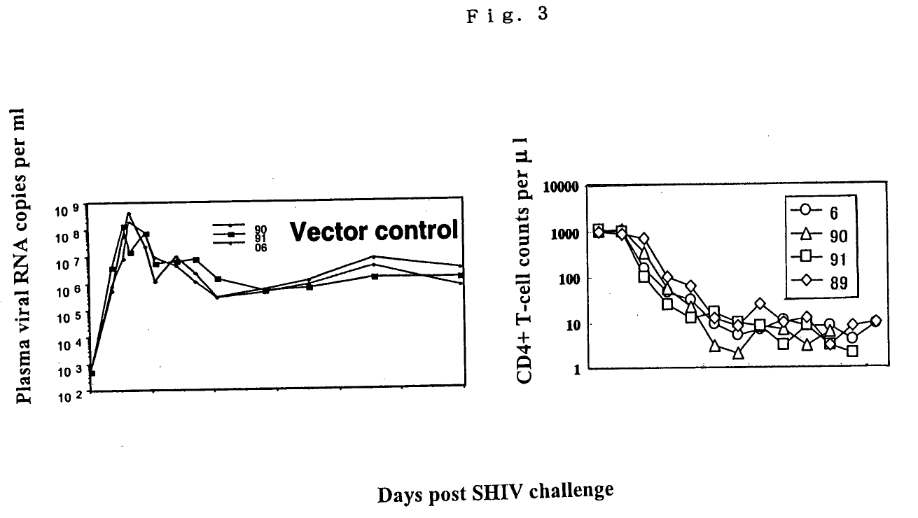

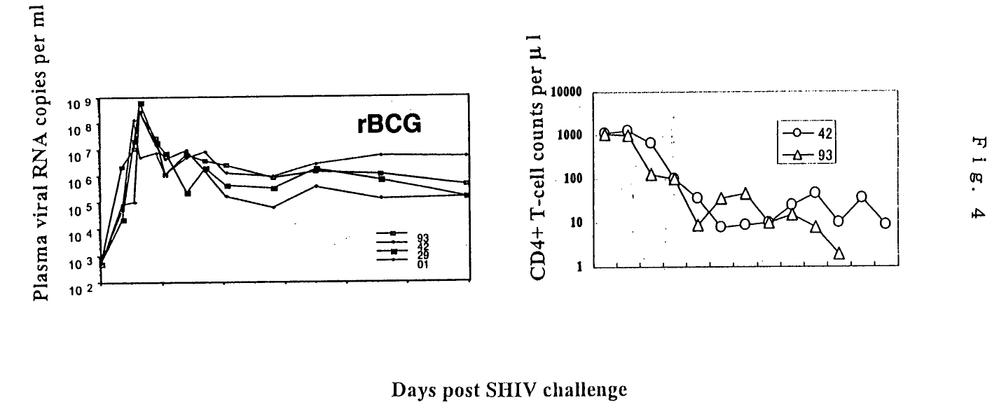

As a result of confirmation of production of SIVgag protein by a Western blotting, a 55 kDa protein was detected in an extract of rBCG-SIVgag as shown in FIG. 2. On the contrary, no Gag protein was detected in rBCG-pSO246 which was a control. Concentration of the SIV Gag protein was 45±12 ng per mg of rBCG-SIVgag and such a productivity level was maintained during at least 450 passages in vitro.

### Example 2

### Immune Induction

Immune induction was carried out in cynomolgus monkey using the recombinant BCG strain prepared in Example 1 (rBCG-SIVgag) and a recombinant vaccinia DIs (rDIs-SIVgag). Incidentally, the rDIs-SIVgag was prepared by the same method mentioned in Example 1 of Japanese Patent Laid-Open No. 2002/017370 using SIVgag instead of HIV-1gag in the said patent.

Fourteen cynomolgus monkeys were divided into the following five groups and immunization (boosting) was carried out at the stage of 0, 47 and 54 week(s) after the initial antigen stimulus.
Group 1 (four macaques): control (one was a naïve macaque while the other three were intracutaneously inoculated with rBCG-pSO246 once and intravenously inoculated with rDIs-LacZ (10⁶ PFU) twice)
Group 2 (two macaques): Intracutaneously inoculated with rBCG-SIVgag (10 mg) once
Group 3 (two macaques): Intravenously inoculated with rDIs-SIVgag (10⁶ PFU) twice
Group 4 (three macaques): Intravenously inoculated with rDIs-SIVgag (10⁶ PFU) twice and intracutaneously inoculated with rBCG-SIVgag (10 mg) once
Group 5 (three macaques): Intracutaneously inoculated with rBCG-pSO246 (10 mg) once and intravenously inoculated with rDIs-LacZ (10⁶ PFU) twice

Then, ten weeks after the second immunity booster, the macaques were challenged with pathogenic virus (SHIV-KS661; 2000 TCID₅₀) via mucous membrane intrarectally, and the change in the number of virus copies and CD4 cell counts in blood were measured periodically.

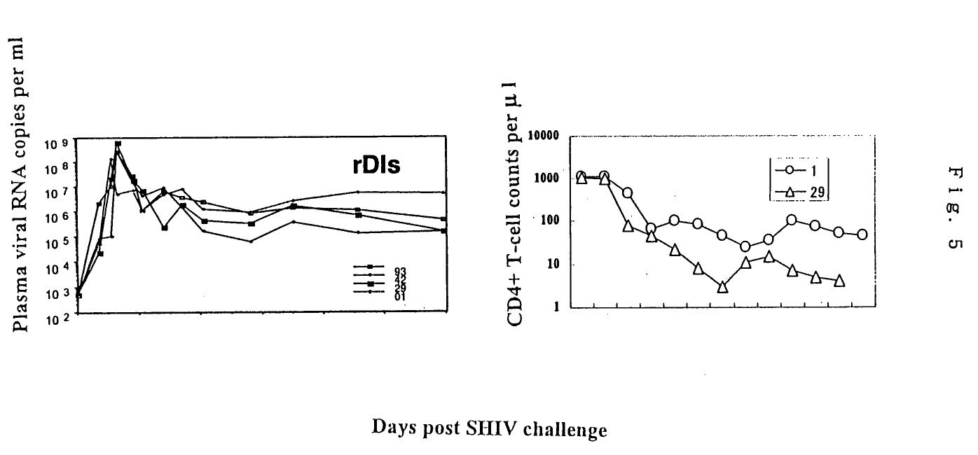

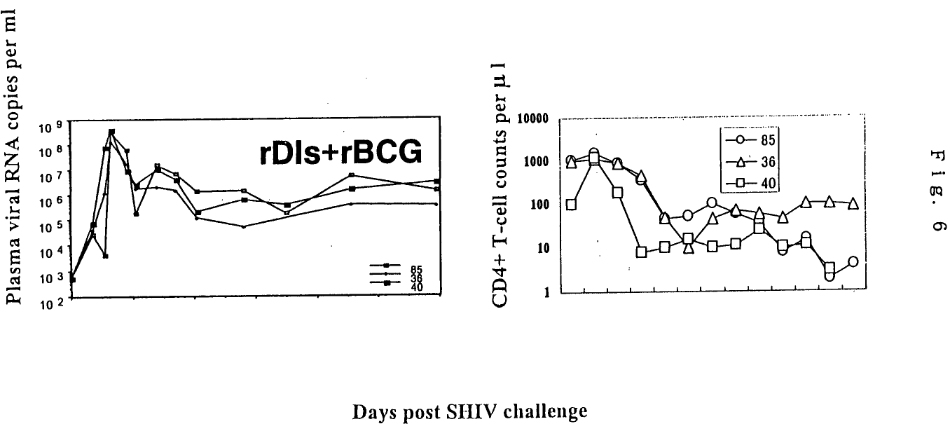

When the macaques of the control group were challenged with SHIV-KS661, CD4 decreased to 1/10 to 1/100 of original levels after about two weeks and, on the other hand, the number of copies of viral RNA in the blood increased by 10⁸⁻⁹, quickly arrived at a set point and shifted to a level of 10⁵⁻⁶. In the case of rDIs-SIVgag only (group 3; FIG. 5), changes in CD4 counts and viral RNA numbers were also as same as those in the case of control. Further, in the case of macaques treated with rDIs-SIVgag (initial antigen stimulus)+rBCG-SIVgag (boosting) (group 4), there was noted the same changes with the lapse of time as in the case of the control and the single immunization.

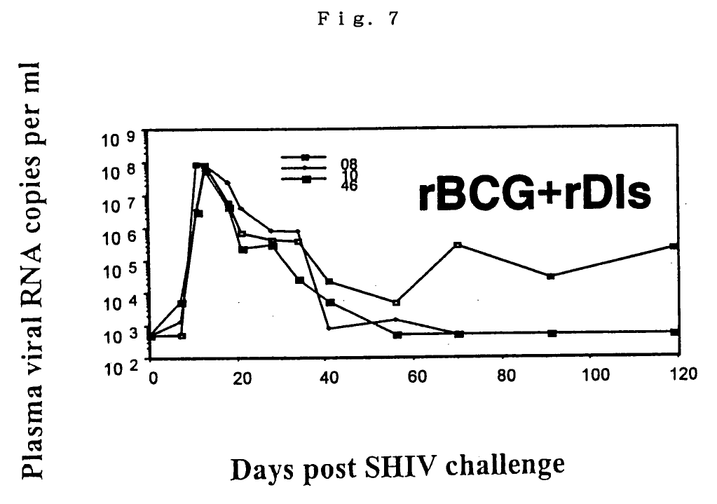

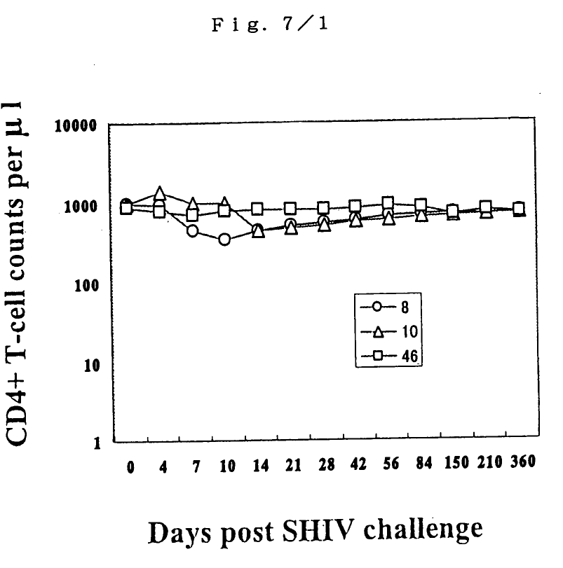

On the contrary, in the case of macaques of group 5 (rBCG-SIVgag+rDIs-SIVgag), the number of copies of viral RNA significantly decreased, indicating that a strong Gag-specific immunity was induced as shown in FIG. 7. Decrease in CD4 cells was also significantly suppressed.

From the above results, it was confirmed that a strong specific immunity was induced when a initial antigen stimulus was conducted using a recombinant BCG vaccine (rBCG-SIVgag) and boosting was carried out using a recombinant DIs vaccine (rDIs-SIVgag).

### Example 3

### Suppression of Anamnestic Reaction of BCG

Influence of the immune induction method of this invention on BCG anamnestic reaction was investigated. Specifically, to simulate influence of administration in a human being, BCG Tokyo strain (0.1 mg) was inoculated to cynomolgus monkeys about two years before and, after confirming that DTH was clearly induced even after two years, vaccine was inoculated to the macaques of the following three groups.
Group 1 (3 macaques): Oral inoculation of rBCG-pSO246 (80 mg) twice+intravenous inoculation of rDIs-LacZ (10⁶ PFU) twice
Group 2 (2 macaques): Oral inoculation of rBCG-SIVgag (80 mg) twice+intravenous inoculation of rDIs-SIVgag (10⁶ PFU) twice
Group 3 (1 macaques): Intravenous inoculation of rBCG-SIVgag (10 mg) once+intravenous inoculation of rDIs-SIVgag (10⁶ PFU) once

The two BCG priming inoculations were carried out at the stage of −48 and 0 week(s) and two boostings were carried out after 27 and 57 weeks. In the group 3, the initial antigen stimulus was conducted at the stage of 0 week and a boosting was conducted at the stage of 57 weeks.

After about three months from the final vaccine inoculation, the macaque was challenged with pathogenic virus (SHIV-C2/1 20TCID₅₀) and the change in CD4 cell counts and viral RNA copy numbers were measured periodically.

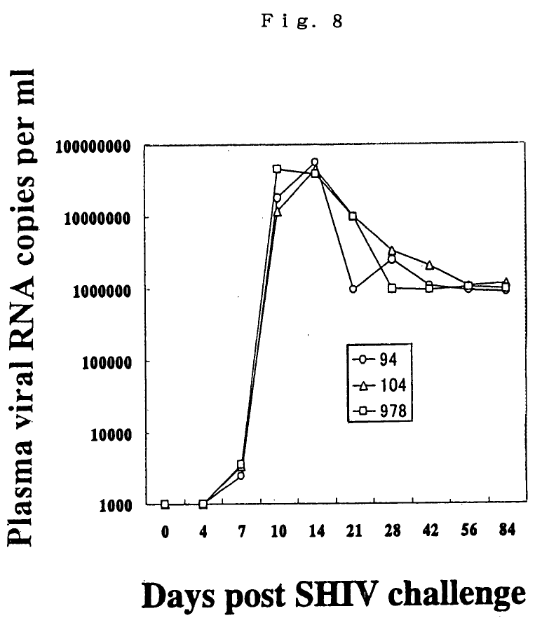

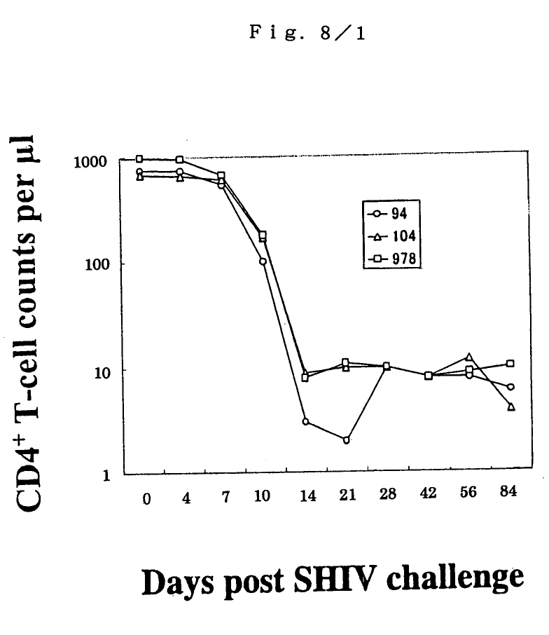

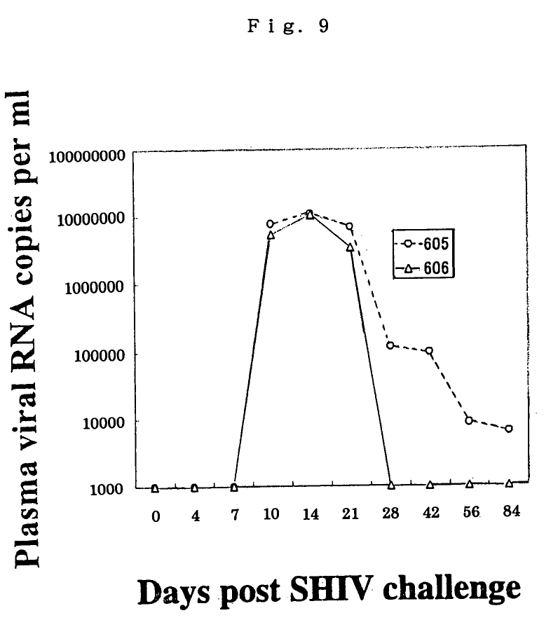

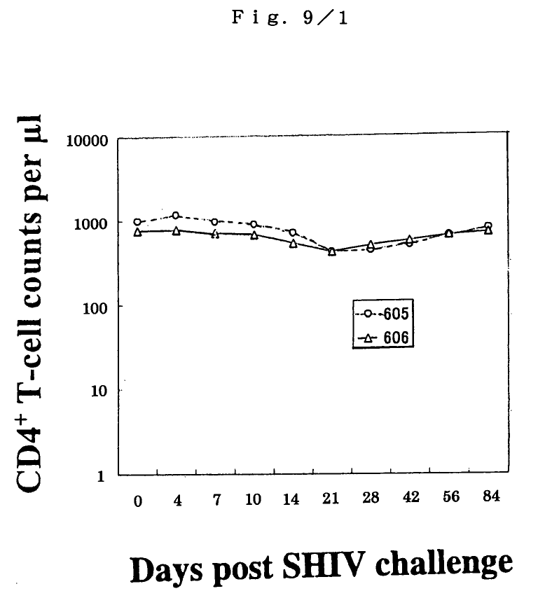

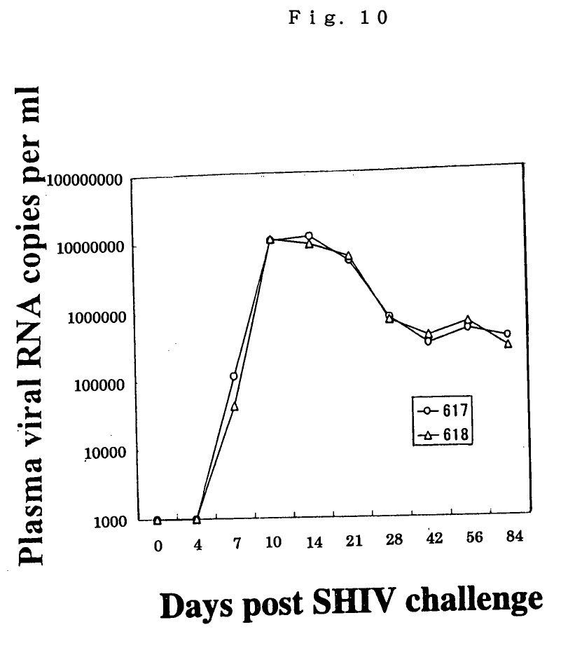

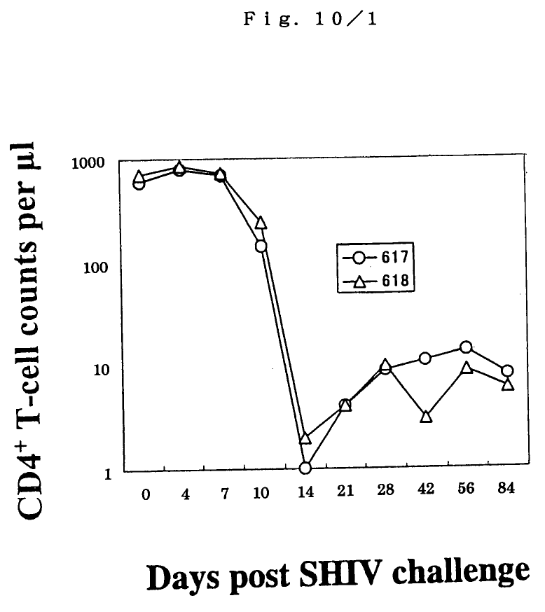

The results are as shown in FIGS. 8 through 10. Changes with the lapse of time of viral RNA numbers and CD4 counts (FIG. 10) by rBCG-SIVgag initial antigen stimulus (intravenous)+rDIs-SIVgag boosting (intravenous) (group 3) were as same as those in the control (group 1). On the contrary, in the case of rBCG-SIVgag initial antigen stimulus (oral) and rDIs-SIVgag boosting (intravenous) (group 2), there were noted a significant decrease in viral amount in blood and a suppression of decrease in CD4 cells.

From the above results, it was confirmed that, when initial antigen stimulus of rBCG-SIVgag was carried out by oral administration and boosting of rDIs-SIVgag was carried out by intravenous inoculation, influence on anamnestic reaction could be excluded not only regarding immune induction but also in protective immunity.

### Industrial Applicability

As fully illustrated hereinabove, an effective immune induction using a recombinant BCG vaccine is now possible in accordance with the invention of this application and an effective prevention of various infectious diseases, cancers, etc. can be achieved.

1. A recombinant BCG vaccine transformed with an expression vector having a polynucleotide encoding an exogenous antigenic protein, which the BCG vaccine is used for initial antigen stimulation in immune induction by plural antigen stimulations.

2. The BCG vaccine according to claim 1, wherein the antigenic protein is derived from immunodeficiency virus.

3. The BCG vaccine according to claim 2, wherein the antigenic protein of immunodeficiency virus is a product of an HIV gene.

4. A method for the immune induction by plural stimulations of exogenous antigenic protein, which comprises carrying out the initial antigen stimulation by the BCG vaccine of claim 1, and carrying out one or more additional antigenic stimulations by non-BCG vaccine expressing the same antigenic protein.

5. The method according to claim 4, wherein the vaccine for the additional antigenic stimulations is a recombinant vaccinia virus vaccine.

6. The method according to claim 4, wherein the antigenic protein is derived from immunodeficiency virus.

7. The method according to claim 6, wherein the antigenic protein of the immunodeficiency virus is an HIV gene product.
